(19) United States
(12) Patent Application Publication
(10) Pub. No.: US 2024/0177120 A1
(43) Pub. Date: May 30, 2024

(54) SYSTEMS AND METHODS FOR MATCHING LEARNERS

(71) Applicant: KONINKLIJKE PHILIPS N.V., EINDHOVEN (NL)

(72) Inventors: Frank BERGNER, HAMBURG (DE); Milosh STOLIKJ, EINDHOVEN (NL); Paul Anthony SHRUBSOLE, ARNHEM (NL)

(21) Appl. No.: 18/515,374

(22) Filed: Nov. 21, 2023

Related U.S. Application Data
(60) Provisional application No. 63/428,452, filed on Nov. 29, 2022.

(51) Int. Cl. G06Q 10/1093; G16H 40/20

(52) U.S. Cl. CPC G06Q 10/1097; G16H 40/20

(57) ABSTRACT

A non-transitory computer readable medium stores at least one database (12) storing an index of training resources (14); and instructions readable and executable by at least one electronic processor (12) to perform a training session generation method (100) includes receiving requests for training from a plurality of medical professionals; obtaining information related to the medical professionals of the plurality of medical professionals; and determining a training session using training resources indexed in the at least one database including determining at least two of the medical professionals to invite to participate in the training session based on the received requests and the obtained information.

### CROSS REFERENCE TO RELATED APPLICATIONS

This application claims the benefit of U.S. Provisional Patent Application Number 63/428,452 filed Nov. 29, 2022. This application is hereby incorporated by reference herein.

### FIELD

The following relates generally to the medical arts, medical education arts, and medical device operations educational content tracking arts, especially as directed to medical imaging devices.

### BACKGROUND

Complex medical devices offer great flexibility in how they can be used to diagnose, monitor, or treat patients. The performance of the medical device may depend strongly on how the operator uses the device, e.g., setting a non-optimal configuration may provide sub-optimal results whereas using a more optimal configuration may provide better results. Moreover, medical devices that are connected to the Internet or another electronic network may receive software or firmware upgrades over the network that provide new features or enhance existing features; however, these may be useless if the operator is not trained to effectively use the new or enhanced features. Thus, there is substantial benefit to offering education and support to get the best results from the medical devices according to the clinical needs of patients and according to the specializations, way of working of the staff, and the type of hospital or clinical practice.

Health care professionals need to be prepared for unfamiliar situations they might be presented with during their daily practice. To do this, they need to learn how to conduct specific procedures, workflows, protocols, or practices for certain clinical cases/patient-situations that are presented to them. Since such highly specific situations or cases occur infrequently, the training and education that they receive is not as effective as it could be because the specific knowledge that was gained about how to deal with such cases will fade over time.

Learning in small groups or pairwise learning can be beneficial in certain scenarios and help to motivate learners and speed up the learning as the learners can jointly discuss and verify their understanding on a clinical topic that is relevant to them at that time. A team lead within a department might want to form learning groups along with a course that multiple staff members are attending.

The following discloses certain improvements to overcome these problems and others.

### SUMMARY

In one aspect, a non-transitory computer readable medium stores at least one database storing an index of training resources; and instructions readable and executable by at least one electronic processor to perform a training session generation method includes receiving requests for training from a plurality of medical professionals; obtaining information related to the medical professionals of the plurality of medical professionals; and determining a training session using training resources indexed in the at least one database including determining at least two of the medical professionals to invite to participate in the training session based on the received requests and the obtained information.

In another aspect, a non-transitory computer readable medium stores at least one database storing an index of training resources; and instructions readable and executable by at least one electronic processor to perform a training session generation method including receiving requests for training from a plurality of medical professionals; obtaining information related to the medical professionals of the plurality of medical professionals; and applying a pairwise comparison cost function to the received requests and the obtained information to determine at least two of the medical professionals to invite to participate in a training session using training resources indexed in the at least one database.

In another aspect, a training session generation method includes receiving requests for training from a plurality of medical professionals; obtaining information related to the medical professionals of the plurality of medical professionals; and applying an artificial intelligence (AI) component to the received requests and the obtained information to determine at least two of the medical professionals to invite to participate in the training session.

One advantage resides in providing targeted grouping of medical professionals for a medical training session.

Another advantage resides in identifying instructors and participants for a medical professional training session.

Another advantage resides in providing targeted grouping of medical professionals for a medical training session based on experience levels and/or credential levels of each medical professional.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically illustrates an illustrative training session generation system in accordance with the present disclosure.

FIG. 2 shows exemplary flow chart operations of the system of FIG. 1.

FIGS. 3, 4, and 5 schematically show operations performed by the system of FIG. 1 in accordance with various embodiments described herein.

### DETAILED DESCRIPTION

Medical professionals in various roles (doctors, oncologists, radiologists, anesthesiologists, nurses, medical imaging technologists, and so forth) engage in frequent training sessions to keep up to date on the latest medical developments, medical equipment, medical regulations, and the like. Conducting training sessions for groups of medical professionals is more efficient than training individuals in isolation. However, group training can have reduced efficacy if the group is too heterogeneous, as various parts of the training content may be of limited or no use to many members of the group. For example, if the group includes junior medical professionals and highly experienced medical professionals, content of a training session tailored to the junior medical professionals may not be useful for the experienced medical professionals of the group; conversely, if the content is tailored to the highly experienced medical professionals of the group that content may be too difficult for the junior medical professionals. As another illustrative example, if the content of the group training session is directed to a particular medical procedure, then the training session may be of little interest to participant medical professionals whose work situation makes it unlikely they will perform that particular medical procedure in the near future. Another issue can arise if the training session is intended to simulate a medical procedure, such as a surgical operation. In such a training session, it would be useful for the participants to encompass all the roles of the surgical procedure (e.g., surgeon, nurse, anesthesiologist, et cetera) so that the surgical procedure simulation can be as complete and realistic as reasonably feasible. As yet another illustrative example, if the training session is to be conducted by an instructor, then it would be beneficial for the learners (i.e., the medical professionals receiving the training) to consider the instructor to be effective in that role. A specific training need may also arise when the medical professional needs training around a patient case, and/or a clinical context or medical use case that isn't covered by standard trainings. In this case, those professionals that have such a similar need can be grouped into the same training session in an ad-hoc fashion.

The following discloses approaches for generating personalized group training recommendations. To do so, requests for training are received. Such requests can explicitly define the desired content of the training or can implicitly define the desired content. As an example of the latter, a doctor may input a request for help with a current patient and the system accesses the patient medical record to determine training content that may assist the doctor with that patient. As another example of the latter, an imaging technologist may request training on a new medical imaging device and the system compares that new device with medical imaging devices on which the technologist is already qualified or experienced to identify features of the new medical device for which training may be helpful.

Information about the medical professionals making the requests is also collected, such as their medical roles, medical experience, geographical locations, language preferences, availability of communication resources (e.g., videocall capabilities), and so forth. This information can be extracted from human resources (HR) records and possibly other sources such as work logs. Optionally, medical professionals using the training session recommender may fill out questionnaires providing some of this information.

Based on the above information, a matching algorithm is applied to identify a group for a training session. In some embodiments, the content of the training session is decided a priori, and the matching identifies medical professionals to invite to the training session. In other embodiments, the content of the training session itself may be part of the optimization, e.g., if there is a sufficiently large group of medical professionals requesting training on aspects of a particular medical procedure then a training session comprising a simulation of that medical procedure may be scheduled based on that information. The following discloses various approaches such as a pairwise comparison cost function or machine learning. By such approaches, a group of two or more medical professionals with a similar training need are identified and assigned to a group training session. Optionally, an electronic calendar is accessed to identify one or more proposed times for the group training session. An invitation to the training session is then issued to the members of the identified group. (In some embodiments, the invitation may be in an “opt-out” invitation, e.g., the training session may be automatically added to the invited participants' electronic calendars, and an invited participant then would need to opt out if desired by declining or removing the training session from the schedule.)

In some embodiments, if the training session requires an instructor, then a similar matching algorithm can be applied to match the most qualified instructor to the group, based on similar factors (e.g., the instructor's experience, areas of expertise, language preferences, et cetera). If instead of an instructor the session is to use a prerecorded video, then a similar approach can be applied to select the most relevant video from a library of training materials.

In a variant of this approach, the information about the common training needs of the group can be used to define the content for a suitable training video which does not yet exist. This variant essentially reverses the application from recommending a group for existing content to recommending content of a video based on the group. (The video would then be manually recorded, based on the recommended content).

In other embodiments, if the training session is to be a simulated medical procedure, then the construction of the group can be modified to ensure that the group includes all roles of the medical team needed to perform the procedure. For example, the group constructed for a simulated medical operation performed by a medical team including a surgeon, two nurses, and an anesthesiologist should include members filling each of those roles.

In some embodiments, if the training session is a group session without an instructor, then it may be useful for the group to have a range of experience levels so that more junior members can benefit from the expertise of more senior members. In this case the matching algorithm may, instead of grouping members with similar experience, rather construct a group whose experience levels fit a desired distribution from junior to senior personnel.

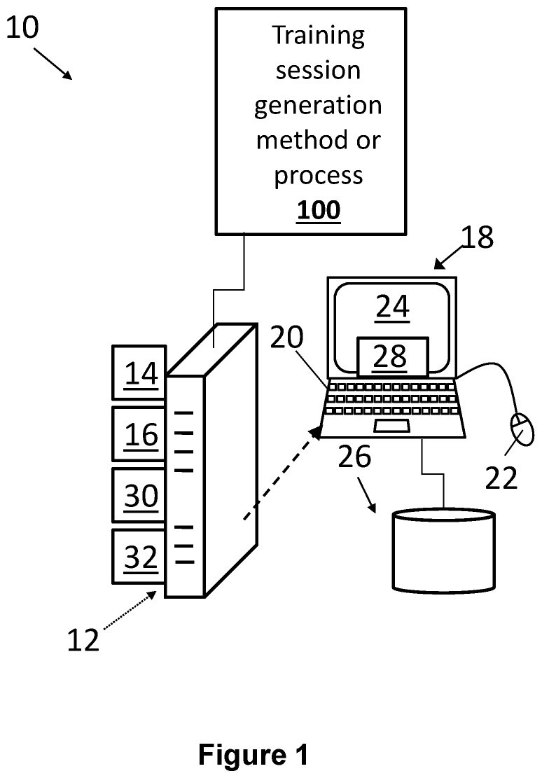

With reference to FIG. 1, an illustrative training session generation system or apparatus 10 for generating a medical procedure training sessions for one or more medical professionals. The apparatus 10 includes, or is in communication with, a server computer 12 storing an index of training resources 14. The index of training resources 14 may index available training content, such as articles, tutorials, videos, or checklists for use by medical professionals when performing medical procedures using a medical device. The index of training resources 14 may additionally or alternatively index other types of training resources, such as available (medical) equipment that can be used in simulation training sessions, available operating rooms for use in simulated medical operation training sessions, available instructors, available training rooms, and/or so forth. Indexed training rooms may be indexed as to availability of specialized equipment such as virtual reality (VR) and/or augmented reality (AR) headsets and an associated presentation system for presenting VR or AR training content. The medical professionals can access the training content or other resources listed in the index 14 during, before, or after a medical procedure. In addition, the server computer 12 can also store medical professional content 16, including one or more of medical roles, medical experience, geographical locations, language preferences, availability of communication resources, credentials of medical professionals who can receive training via the apparatus 10, experience levels of the medical professionals, schedules of the medical professionals, and so forth. This information 16 can be collected in various ways, such as via forms filled out by the medical professionals when enrolling in the training apparatus 10, extracted from human resources (HR) records, accreditation records, work records, and/or so forth.

FIG. 1 also shows, an electronic processing device 18, such as a workstation computer, a tablet, or more generally a computer. Additionally or alternatively, the electronic processing device 18 can be embodied as a server computer or a plurality of server computers, e.g., interconnected to form a server cluster, cloud computing resource, or so forth. The electronic processing device 18 includes typical components, such as an electronic processor 20 (e.g., a microprocessor), at least one user input device (e.g., a mouse, a keyboard, a trackball, and/or the like) 22, and at least one display device 24 (e.g., an LCD display, plasma display, cathode ray tube display, and/or so forth). In some embodiments, the display device 24 can be a separate component from the workstation 12. The display device 24 may also comprise two or more display devices. The electronic processor 20 is operatively connected with a one or more non-transitory storage media 26. The non-transitory storage media 26 may, by way of non-limiting illustrative example, include one or more of a magnetic disk, RAID, or other magnetic storage medium; a solid state drive, flash drive, electronically erasable read-only memory (EEROM) or other electronic memory; an optical disk or other optical storage; various combinations thereof; or so forth; and may be for example a network storage, an internal hard drive of the electronic processing device 18, various combinations thereof, or so forth. It is to be understood that any reference to a non-transitory medium or media 26 herein is to be broadly construed as encompassing a single medium or multiple media of the same or different types. Likewise, the electronic processor 20 may be embodied as a single electronic processor or as two or more electronic processors. The non-transitory storage media 26 stores instructions executable by the at least one electronic processor 20. The instructions include instructions to generate a graphical user interface (GUI) 28 for display on the display device 24.

The server computer 12 stores instructions executable by the server computer 12 to perform a training session generation method or process 100 implemented by the system 10 In some examples, the method 100 may be performed at least in part by cloud processing (that is, the server computer 12 may be implemented as a cloud computing resource comprising an ad hoc network of server computers).

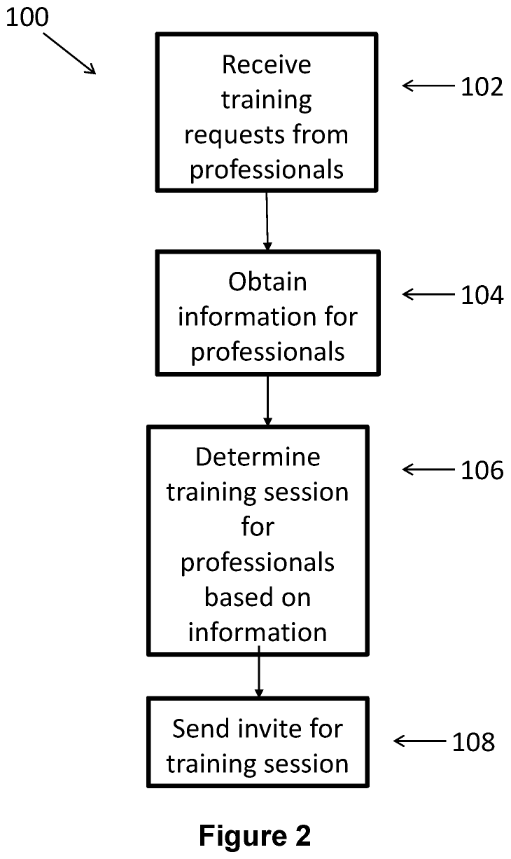

With reference to FIG. 2, and with continuing reference to FIG. 1, an illustrative embodiment of an instance of the method 100 is diagrammatically shown as a flowchart. To begin the method 100, the GUI 28 is provided on the display device 24 of the electronic processing device 18. At an operation 102, a request for a training session by a plurality of medical professionals can be received at the server computer 12. Such training requests can explicitly define the desired content of the training or can implicitly define the desired content. For example, one or more doctors may input a request for help with a current patient, and the desired content of the training can be extracted from the medical records of that patient or from information about the patient entered as part of the request. As a nonlimiting illustrative specific example, if the request is from a cardiac perfusionist for assistance with a cardiac procedure using a newly acquired cardiopulmonary bypass machine (CPB machine, also known as a heart-lung machine), then this request may implicitly define a request for training on the new CPB machine. In another example, one or more imaging technologists may request training on a new medical imaging device.

At an operation 104, information related to the medical professionals of the plurality of medical professionals (i.e., the medical professional content 16) can be obtained from the server computer 12.

At an operation 106, a training session is determined and generated for at least two of the medical professionals who submitted training requests based on the medical professional content 16. In one example embodiment, a pairwise comparison cost function 30 (stored in the server computer 12) is executed by the server computer 12 and applied to the received training requests and the obtained medical professional content 16 to determine a training need for each of the medical professionals who submitted a training request. At least two of the medical professionals having a common training need (i.e., two technologists submitted a request for training on a new imaging device) are determined. In another example, instead of the pairwise comparison cost function 30 an artificial intelligence (AI) component 32 (i.e., an artificial neural network (ANN)) (stored in the server computer 12) is executed by the server computer 12 and applied to the received training requests and the obtained medical professional content 16 to determine a training need for each of the medical professionals who submitted a training request. Training resources listed in the index 14 to be used in the training session can also be determined.

In some embodiments, the training need addressed by the training session is known a priori, and the operation 106 determines the two or more medical professionals to invite to the training session. In other embodiments, the operation 106 itself determines the training need that the training session is to fulfill (in effect, creating the training session itself). As an example of the latter, the operation 106 may identify when at least N requests have been received for a particular training need, and once N requests have been received for training on that training need the system schedules one (or two, or more) training sessions directed to that training need. If two or more such sessions are scheduled, then the distribution of the medical professionals requesting that training need can be automatically distributed amongst the training sessions. In variant embodiments, other factors can be used to decide when to schedule a training session (or multiple sessions) directed to a given training need, such as indications in the requests of urgency or time constraints on the training (e.g., if some requests indicate the training must be completed by end-of-year to satisfy a credentialing requirement then this may cause the system to schedule one or more training sessions ahead of that end-of-year deadline).

In some examples, roles for a medical procedure of the training session for each of the medical professionals participating in the training session can be determined. For example, for a medical procedure training session, one of the participants can be assigned as a “patient” during the training session, another participant can be assigned as a “doctor” during the training session, another participant can be assigned as a “nurse” during the training session, and so forth.

In another example, experience levels for each of the medical professionals who submitted the request for training can be determined, and the medical professionals can be determined for a training session according to a predetermined distribution of experience levels. For example, it may be useful for the training session to have a range of experience levels so that more junior members can benefit from the expertise of more senior members during the training session.

In another example, medical cases which are new to the medical professionals can be determined, and the medical professionals can be determined for a training session according to the medical cases which are new to the medical professionals.

In another example, an instructor from a plurality of potential instructors for the training session can be determined based on the common training need and information related to the instructors. For example, credentials, experience, and other suitable data of a pool of instructors can be used to identify the “best” instructor for the training session.

At an operation 108, an invitation is output (i.e., via email, text message, etc.) to the determined medical professionals for the training session. To do so, a calendar for each determined medical professional is accessed to find a common time for the training session to occur. In addition to accessing the electronic calendars for the invited learners, this may also entail accessing electronic calendars for resources to be utilized in the training session. For example, if the training session requires a VR-equipped room then the electronic calendar system may include a calendar indicating when the VR-equipped room is available, and this information is also used to ensure the training session is scheduled at a time when both (1) all learners are available and (2) the VR-equipped room is available.

### EXAMPLE

The following provides another example of the training session generation system or apparatus 1010. The apparatus 10 comprises an assistant system that tries to find optimized combinations of learners and potentially trainers. Input to the apparatus 10 are credentials of the participants. The apparatus 10 uses defined comparison functions for the individual learner properties and generates a cost function that can be optimized mathematically. The optimization results are then proposed to the person that schedules pair learnings or courses, which can also be the learner. The system searches for possible free time slots and rooms (if non-virtual pair learnings are preferred) and shows these as selectable options.

In the operation of the apparatus 10, certain learner aspect might be more or less important. The uses of the apparatus 10 can choose and tuning a weighting for the individual learner aspects to influence the optimization in this direction.

Part of the apparatus 10 can be a continuous loop of updating the estimated experience level of the learner as learning progress can change dynamically through learning journey. For this purpose, the apparatus 10 can include to take updated inputs from a learning progress estimation system. Such a system can take inputs from learning assessments (i.e., quizzes) but can also take into account real live application of the learnings (i.e., being tracked in a learning record store (LRS)). The estimated learning level can then be considered, and updated, so more optimal learning groups can be formed.

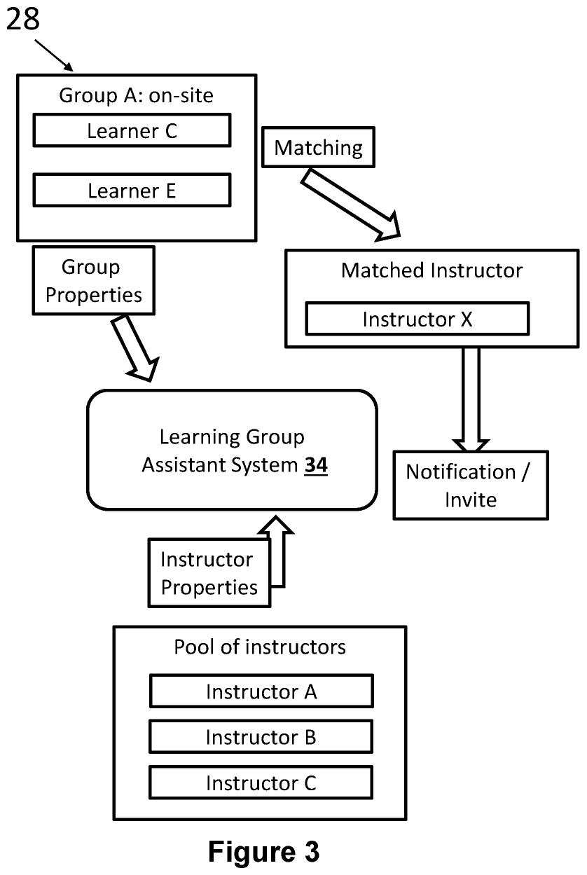

As shown FIG. 3, a simplified illustration of the GUI 28 is depicted. The user first selects a set of eligible learners that need to be scheduled to learning groups. This list is passed to an assistant system 34 along with the individual user properties. The user can then select a weighting for certain optimization criteria, for example, that groups should be formed from learners with roughly the same experience level. Based on this the optimization is done and the user is presented an optimized set of different learning groups.

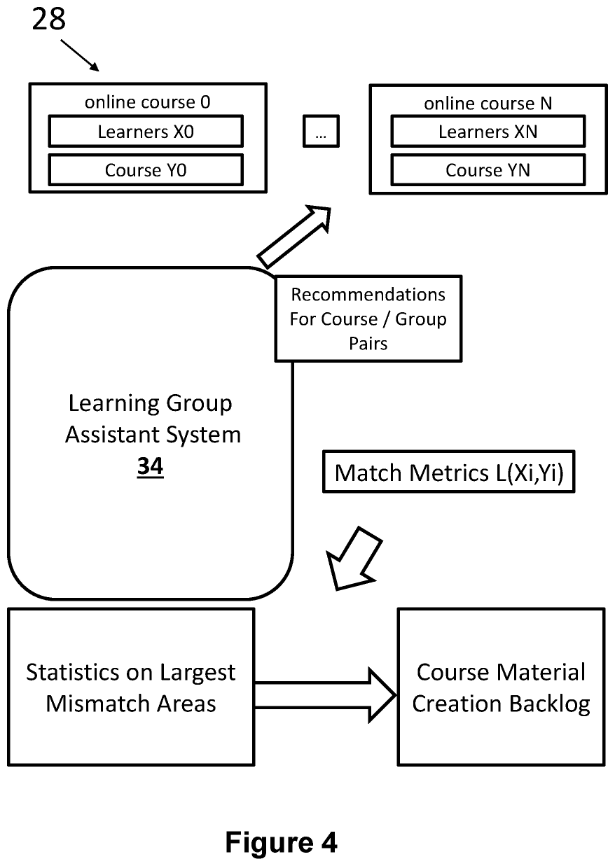

The user is then also presented potential options for the time-slots for a learning group, where the learner and potentially also the learning rooms, instructors, or other scarce resources necessary for the courses are available for the group learning activity. This is illustrated in FIG. 4.

In one embodiment the apparatus 10 is built from individual comparison functions dᵢ(x_{i,k}, x_{i,j}) that indicates a mismatch (high value) in an i-th property, where x_{i,k} is the i-th property that belongs to the k-th learner and x_{i,j} belongs to the j-th learner. The first optimization function is created by using a weighted sum of all occurring weighting function from all learners and properties according to Equation 1:

$$L ( k , j ) = {\sum }_{i} {w}_{i} {d}_{i} ( {x}_{i , k} , {x}_{i , j} )$$

where wᵢ are selectable, scalar weights that can be chosen by the user. This type of cost function indicates how well two learners fit to each other.

In some embodiments, the apparatus 10 now tries different learner combinations and finds the set of combinations that satisfy a global optimization criterium, for instance, that the total sum of occurring L (k, j) for all groups is minimal. This would mean that the total mismatch in learners is minimized. Other optimization criteria are possible, e.g., trying to avoid that mismatched become too high.

In another embodiment certain user properties might be too abstract and forming quantitative mismatch function can be difficult. Yet, historic information about learning groups and ratings out their effectiveness, e.g., quiz results or user surveys, are available. Based on the historic data a machine learning algorithm is trained that tries to predict the usefulness of a certain learner combination, and therefore replaces the direct modelling of a mismatch function. Instead, the machine learning model is then used as a surrogate for the cost function.

In another embodiment, geography and language preferences are factored into the function such that group pairings are possible virtually across geographies or even languages using a real-time language translation functionality (speech to text or speech to text to speech using a slight delay in the video feed with lip synching).

In another embodiment the availability and compatibility of virtualization hardware is taken into account in the cost function such that, if a learner has a virtual reality system, augmented reality system or simulation access, they can be paired up at a lower cost compared to other learners.

In another embodiment learners are matched not only by role but also the patient and disease types they are exposed to in their work. The system analyses the past experience of the learner in this regard, i.e., by comparing past activities and EMR records of the patients involved, and creating new, descriptive properties for the comparison function for the given user. Examples can be working with children vs. working with elderly patients or working in cardiac interventions vs. neurologic interventions.

In another embodiment, learners that have upcoming patients with similar properties (e.g., comorbidities, similarity to particular case study recently presented by an instructor, treatments etc.), are placed in a common group. This group is scheduled for virtual or on-site session, given geographical feasibility and equipment availability.

In another embodiment, learners that have not treated a patient with certain property (e.g., comorbidities, treatment etc.), and are not expected to in the near future (based on the hospital scheduling system), but need to, given their seniority/certification requirements/expressed interest, are paired in a common group. The group is matched to an instructor with expertise in the given area and assigned a physical or virtual location for training.

In another embodiment, the course content is automatically updated.

In another embodiment, a pool of instructors or experienced physicians are made available via an online/remote learning platform. Once a particular set of criteria are met when a learning groups, the most closely matched instructors will be notified so that scheduling can be initiated.

In another embodiment, online learner-based requests are analysed and matched to available digital learning assets. If none of the available assets sufficiently match the request (patient context, medical device context, learner context), then a pool of subject matter experts who are subscribed to the digital learning platform are notified so that they may modify or develop new learning content. If so, then the matched learners are notified, and the learning activity is scheduled based on the ETA of the content.

In another embodiment, the digital learning system provides learners with a means to ask questions about how to deal with particular patient cases or particular clinical research studies using natural language. As well as providing immediate results using a natural-language based search engine (the results of which would include prior responses and existing materials/guidelines), the system also analyses at a statistical level the search queries to match learners with similar experience levels and learning needs for future collaborative learning recommendations. Other embodiments can involve the publication of a new medical procedure or technology for which learners who currently qualify can be grouped.

In the previous embodiments, the index of training resources 14 stores indexes training content. However, as previously noted, the index of training resources 14 may index other types of training resources, such as available equipment for use in training, available operating rooms, available training rooms (possibly VR or AR equipped), and/or so forth.

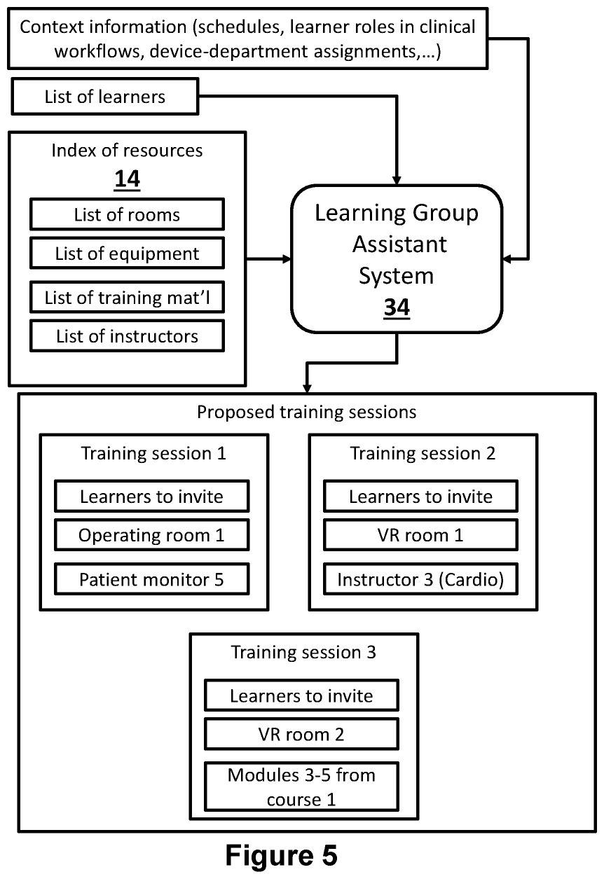

With reference to FIG. 5, a further example of the learning group assistant system 34 is shown which generates a training session using such information. The input information includes the list of learners as previously discussed, e.g., identified from the received requests for training, and the index of resources 14 including a list of training materials but also including a list of available training rooms, a list of equipment for use in training, and a list of available instructors. The input may also include contextual information such as requestor and instructor schedules (from an electronic calendar system, for example), learner roles, clinical workflows (useful for scheduling a clinical procedure simulation training session, for example), device department assignments (for example, a given patient monitor may only be available for training of requestors from within a department that owns that patient monitor), and/or so forth. As seen in FIG. 5, the output training sessions include the learners to invite and other resources to be utilized by the training sessions. For example, “Training session 1” includes an allocated “Operating room 1” and an allocated “Patient monitor 5” to be used in a training session that simulates a medical procedure that employs that patient monitor. “Training session 2” is performed using virtual reality (VR) and hence includes an allocation of “VR room 1” as well as a cardiac “Instructor 3” who is to lead the VR-based cardiac procedure simulation training. “Training session 3” is also a VR training session and utilizes VR content “Modules 3-5 from course 1”. These are merely nonlimiting illustrative examples.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A non-transitory computer readable medium storing:
at least one database storing an index of training resources; and
instructions readable and executable by at least one electronic processor to perform a training session generation method comprising:
receiving requests for training from a plurality of medical professionals;
obtaining information related to the medical professionals of the plurality of medical professionals; and
determining a training session using training resources indexed in the at least one database including determining at least two of the medical professionals to invite to participate in the training session based on the received requests and the obtained information.

2. The non-transitory computer readable medium of claim 1, wherein the instructions further include:
outputting an invitation to the at least two of the medical professionals for the determined training session.

3. The non-transitory computer readable medium of claim 1, wherein the determining includes:
applying a pairwise comparison cost function to the received requests and the obtained information to determine the at least two of the medical professionals to invite to participate in the training session.

4. The non-transitory computer readable medium of claim 1, wherein the determining includes:
applying an artificial intelligence (AI) component to the received requests and the obtained information to determine the at least two of the medical professionals to invite to participate in the training session.

5. The non-transitory computer readable medium of claim 3, wherein the determining includes:
accessing a calendar for the at least two medical professionals to determine a time for the training session.

6. The non-transitory computer readable medium of claim 3, wherein the instructions further include:
determining an instructor for the training session from a plurality of potential instructors for the training session based on the obtained information related to the at least two medical professionals and information related to the potential instructors.

7. The non-transitory computer readable medium of claim 3, wherein the determining includes:
determining the training resources used in the training session based on the received requests and the obtained information about the medical professionals of the plurality of medical professionals.

8. The non-transitory computer readable medium of claim 3 wherein the determining includes:
determining roles of a medical procedure to be practiced in the training session for each of the at least two medical professionals participating in the training session based on the obtained information about the at least two medical professionals.

9. The non-transitory computer readable medium of claim 1, wherein the obtained information includes experience levels for the medical professionals and the determining includes:
determining the at least two medical professionals to invite to participate in the training session according to a predetermined distribution of experience levels.

10. The non-transitory computer readable medium of claim 1, wherein the information related to the medical professionals includes one or more of medical roles, medical experience, geographical locations, language preferences, and availability of communication resources.

11. A non-transitory computer readable medium storing:
at least one database storing an index of training resources; and
instructions readable and executable by at least one electronic processor to perform a training session generation method comprising:
receiving requests for training from a plurality of medical professionals;
obtaining information related to the medical professionals of the plurality of medical professionals; and
applying a pairwise comparison cost function to the received requests and the obtained information to determine at least two of the medical professionals to invite to participate in a training session using training resources indexed in the at least one database.

12. The non-transitory computer readable medium of claim 11, wherein the instructions further include:
outputting an invitation to the at least two of the medical professionals for the determined training session.

13. The non-transitory computer readable medium of claim 11, wherein the determining includes:
accessing a calendar for the at least two medical professionals to determine a time for the training session.

14. The non-transitory computer readable medium of claim 11, wherein the instructions further include:
determining an instructor for the training session from a plurality of potential instructors for the training session based on the obtained information related to the at least two medical professionals and information related to the potential instructors.

15. The non-transitory computer readable medium of claim 11, wherein the determining includes:
determining the training resources used in the training session based on the received requests and the obtained information about the medical professionals of the plurality of medical professionals.

16. The non-transitory computer readable medium of claim 11, wherein the determining includes:
determining roles of a medical procedure to be practiced in the training session for each of the at least two medical professionals participating in the training session based on the obtained information about the at least two medical professionals.

17. The non-transitory computer readable medium of claim 11, wherein the obtained information includes experience levels for the medical professionals and the determining includes:
determining the at least two medical professionals to invite to participate in the training session according to a predetermined distribution of experience levels.

18. The non-transitory computer readable medium of claim 11, wherein the information related to the medical professionals includes one or more of medical roles, medical experience, geographical locations, language preferences, and availability of communication resources;
wherein the determining includes determining the at least two medical professionals to invite to participate in the training session according to medical cases which are new to the medical professionals.

19. A training session generation method, comprising:
receiving requests for training from a plurality of medical professionals;
obtaining information related to the medical professionals of the plurality of medical professionals; and
applying an artificial intelligence (AI) component to the received requests and the obtained information to determine at least two of the medical professionals to invite to participate in the training session.

20. The method of claim 19, further including:
outputting an invitation to the at least two of the medical professionals for the determined training session.
